(19) United States
(12) Patent Application Publication
(10) Pub. No.: US 2023/0410961 A1
(43) Pub. Date: Dec. 21, 2023

(54) SUBSCRIPTION-BASED ORAL HEALTH NETWORK

(71) Applicant: 3M INNOVATIVE PROPERTIES COMPANY, St. Paul, MN (US)

(72) Inventors: Frederick D. Denny, St. Paul, MN (US); Jay G. Issa, Woodbury, MN (US); David K. Cinader, Jr., Woodbury, MN (US); Amy L. Unklesbay, Stillwater, MN (US)

(21) Appl. No.: 18/030,569

(22) PCT Filed: Oct. 29, 2021
(86) PCT No.: PCT/IB2021/060047

§ 371 Date: Apr. 6, 2023

Related U.S. Application Data
(60) Provisional application No. 63/109,143, filed on Nov. 3, 2020.

(51) Int. Cl. G16H 10/60; G06Q 20/12

(52) U.S. Cl. CPC G16H 10/60; G06Q 20/127

(57) ABSTRACT

The present disclosure provides a subscription-based oral health care network that is patient-focused and tied to a patient dental health record (DHR) that can be shared amongst network participants. The network may include many service providers including treating professionals (e.g., doctors, dentist, orthodontists, etc.), retail partners, product suppliers, insurers, oral health experts, logistics and shipping entities, and dental appliance manufacturers.

### BACKGROUND

Orthodontics is a field of dentistry associated with the professional supervision, guidance and correction of malpositioned teeth. The benefits of orthodontic treatment include attaining and maintaining a proper bite function, enhancing facial aesthetics, and improving dental hygiene. To achieve these goals, the orthodontic professional often makes use of corrective appliances that engage to the patient's teeth and apply gentle therapeutic forces to move the teeth toward proper positions.

One common type of treatment uses tiny, slotted appliances called orthodontic brackets, which are adhesively attached to either the front or back surfaces of the teeth. To begin treatment, a resilient arch-shape wire (“archwire”) is received into the slot of each bracket. The ends of the archwire are generally captured in appliances called molar tubes, which are affixed to the patient's molar teeth. As the archwire slowly returns to its original shape, it acts as a track that guides the movement of teeth toward desired positions. The brackets, tubes, and archwire are collectively known as “braces.”

Orthodontic treatment may also involve the use of alignment trays, such as clear or transparent, polymer-based tooth positioning trays, often referred to as clear tray aligners (CTAs). For example, orthodontic treatment with CTAs may include forming a tray having shells that engage one or more teeth. Each shell may be deformed from an initial position of a tooth, e.g., a malocclusion position. The deformed position of a respective shell of the CTA may apply a force to a respective tooth toward a desired position of the tooth that is an intermediate position between the initial position and a final position resulting from the orthodontic treatment. In some examples, small attachments may be bonded to the teeth to improve force application or achieve desired tooth movements. Proper placement of attachments may ensure proper engagement and interaction of the attachment with a designed feature on the CTAs. The designed feature may provide a desired physical leverage which creates a desired force on a tooth to produce a specific movement of the tooth during treatment. Attachments are typically constructed of varying materials, shapes and sizes, and can be bonded to the labial or lingual surfaces of teeth in order to interact with CTAs and removable appliances in a variety of different ways. Attachments can be applied to a patient's teeth prior to treatment with aligners. Attachments may also be fabricated prior to tooth attachment. Attachments may also be substantially assembled at the orthodontic practitioner's office prior to, or in conjunction with, positioning on the patient's tooth (e.g., molded composites, etc.).

Restorative dentistry may be used to add tooth structure to a patient's dentition, e.g., to an existing tooth, in order to improve at least one of function, integrity, aesthetics, or morphology of missing or irregular tooth structure. For example, restorative dentistry can be an aesthetic treatment to improve appearance of teeth by, for example, altering their shape and/or optical properties (e.g., shade, translucency), which can be achieved using any suitable technique, such as by applying a veneer, managing position or contour of adjacent soft tissues, lessening or removing a gap (diastema) and/or resolving the appearance of malposition. As another example, restorative dentistry may be used to adjust the biting or chewing function of teeth to affect tooth function and/or other aspects of overall oral health such as temporomandibular joint (TMJ) disorders, excessive wear, periodontal involvement, gingival recession or as part of a larger plan to construct a healthy and stable oral environment. Restorative dentistry may be performed in complementary fashion to orthodontic treatment.

### SUMMARY

The present disclosure provides a subscription-based oral health care network that is patient—focused and tied to a patient dental health record (DHR) that can be shared amongst network participants. The network may include many service providers including treating professionals (e.g., doctors, dentist, orthodontists, etc.), retail partners, product suppliers, insurers, oral health experts, logistics and shipping entities, and dental appliance manufacturers. The service providers may opt-in to the network for visibility or pay subscription fees based on, e.g., the number of patients referred or treated. The network supports a virtual community of dental patients, dentists, specialists such as orthodontists and oral surgeons, financial institutions, benefit providers and the providers of dental equipment or services. For treating professionals, such as dentists and orthodontists, the system provides a solution for planning patient treatments, managing communication with patients, storing patient records and sharing patient records with relevant persons outside the doctor's office, including for example, consulting professionals or equipment manufacturers. For patients, the system provides a broad array of dental-care resources that help consumers find answers to their critical dental questions and make informed purchasing decisions. The system is convenient to use and provides informative shopping experience through which dental care services and dental-related products can be distributed. Consumers can access the system using a personal computing device at any time, conveniently from anywhere network access is available. A customer can store his or her dental history and other relevant dental information, as well as share oral health and dental treatment goals with network participants. The patient can thus control the creation of and access to a centralize record of his or her oral health.

### BRIEF DESCRIPTION OF THE DRAWINGS

FIG. 1 is a diagram of an oral health subscription network structure according to embodiments of the present disclosure;

FIG. 2 is a diagram of an aspect of the oral health subscription network of FIG. 1, exploring an entrance tier according to embodiments of the present disclosure;

FIG. 3 is a diagram of another aspect of the oral health subscription network of FIG. 1, exploring an enhanced tier according to embodiments of the present disclosure;

FIG. 4 is a diagram of another aspect of the oral health subscription network of FIG. 1, exploring a premium tier with monthly fees according to embodiments of the present disclosure;

FIG. 5 is a diagram of another aspect of the oral health subscription network of FIG. 1, exploring a premium tier with treatment stage based fees according to embodiments of the present disclosure;

FIG. 6 is a flowchart of a process for interacting with the oral health subscription network of FIG. 1 to select a treatment, from the perspective of a patient;

FIG. 7 is a flowchart of a process for providing the treatment(s) select in the process of FIG. 8 to the patient; and

FIG. 8-9 illustrate example devices that can be used in the embodiments shown in previous Figures.

Features and advantages of the present disclosure will be further understood upon consideration of the detailed description as well as the appended claims.

### DETAILED DESCRIPTION

The sections below describe illustrative embodiments directed to an oral health subscription network and methods related thereto. These embodiments are exemplary and accordingly should not be construed to unduly limit the invention. For example, it is to be understood that one of ordinary skill can adapt the disclosed workflows and methods for myriad dental appliances.

The functions or algorithms described herein may be implemented in software in one embodiment. The software may consist of computer executable instructions stored on computer readable media or computer readable storage device such as one or more non-transitory memories or other type of hardware based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine. The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

Often, computer readable media are provided as part of a computing device. The computing device may have one or more processors, volatile memory (RAM), a device for reading machine-readable media, and input/output devices, such as a display, a keyboard, and a pointing device. Further, a computing device may also include other software, firmware, or combinations thereof, such as an operating system and other application software. A computing device may be, for example, a workstation. a laptop, a tablet, a smart phone, a personal digital assistant (PDA), a server, a mainframe or any other general-purpose or application-specific computing device. A computing device may read executable software instructions from a computer-readable medium (such as a hard drive, a CD-ROM, or a computer memory), or may receive instructions from another source logically connected to computer, such as another networked computer. Data can be communicated directly to an application, e.g., on a mobile device and/or directly to a cloud platform system via cellular connection, a Wi-Fi router or a hub.

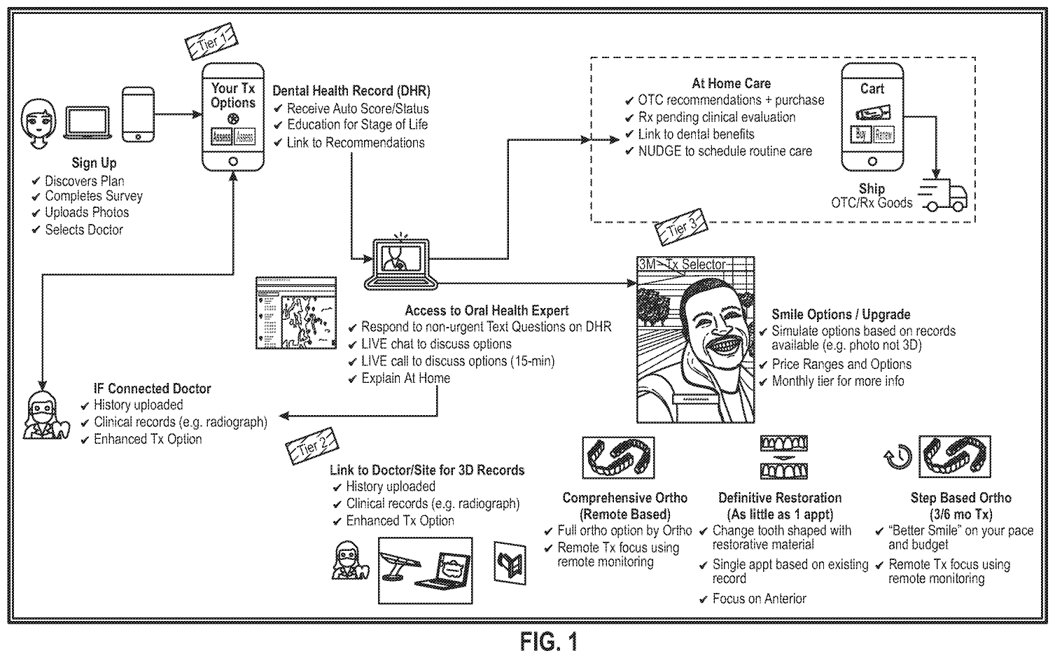

An exemplary oral health subscription network is illustrated in FIG. 1. The network is patient—focused and tied to a patient dental health record (DHR) that can be shared amongst network participants. The network may include many service providers including treating professionals (e.g., doctors, dentist, orthodontists, etc.), retail partners, product suppliers, insurers, oral health experts, logistics and shipping entities, and dental appliance manufacturers. The service providers may opt-in to the network for visibility or pay subscription fees based on, e.g., the number of patients referred or treated.

The network supports a virtual community of dental patients, dentists, specialists such as orthodontists and oral surgeons, financial institutions, benefit providers and the providers of dental equipment or services. For treating professionals, such as dentists and orthodontists, the system provides a solution for planning patient treatments, managing communication with patients, storing patient records and sharing patient records with relevant persons outside the doctor's office, including for example, consulting professionals or equipment manufacturers. For patients, the system provides a broad array of dental-care resources that help consumers find answers to their critical dental questions and make informed purchasing decisions. The system is convenient to use and provides informative shopping experience through which dental care services and dental-related products can be distributed. Consumers can access the system using a personal computing device at any time, conveniently from anywhere network access is available. A customer can store his or her dental history and other relevant dental information, as well as share oral health and dental treatment goals with network participants. The patient can thus control the creation of and access to a centralize record of his or her oral health.

A network platform (e.g., a single server, distributed servers, or cloud platform) supports an oral health subscription network with one or more patients, one or more treating professionals, and one or more service providers. The platform includes a processor adapted to communicate with a network; a data storage device coupled to the processor and adapted to store data for each patient; and software to communicate patient data in response to a patient, treating professional, or service provider request.

Through the network, the patient can access a server (e.g., cloud computing platform) that houses dental information through a personal computing device (e.g., smartphone or other mobile device). The network serves as a portal for providing and distributing oral health-related information to interested parties such as dental patients, dentists, orthodontists, dental appliance manufacturers, dental equipment manufactures, dental supply retailers and distributors, and others. When sensitive information is communicated through the subscription network such information may be securely encrypted throughout the transaction. The network can rely on a single server or a cloud computing platform that can distribute processing and communication activity across a computer network.

The network allows patients access to myriad services provided by the participants in the network such as oral health advice and orthodontic treatment, among others. A server or cloud platform may store information associated with patient history (i.e., a DHR) in a secure manner and also provide a comprehensive view of the patient's treatment history at any time using a suitable browser, eliminating the need to pull treatment files or charts or to search for misfiled or lost charts. The network also provides network users with tools to analyze patient data, for example, tools to reconstruct a 3D model or rendering of the patient's teeth. For example, using the network, the patient can request an animation of potential options for orthodontic treatment and simulated results of such treatment. The patient can select a treatment based in part on the animation and be connected with a treating professional to prescribe appliances to effectuate the treatment.

In addition to communicating with patients and treating professionals, the dental server can communicate with one or more partners. The partners can be product suppliers, service providers, or any other suitable commercial entity.

The subscription network can be divided into tiers, with different oral health information and treatment options available in each tier. For instance, a first tier (Tier 1) may include access to a patient portable dental health record (DHR) and oral health care recommendations. The DHR may be filled in via information from the patient or from treating professionals in the network. The DHR may be updated based on actions (e.g., dental treatments) taken by network participants to include new health conditions or patient visual records.

A patient may sign up to the network using existing retail partner (e.g., e-commerce or social media website) credentials (e.g., login name, email and/or password). Alternatively, the patient may create unique account credentials specific to the oral health subscription network. Login screens may be provided to the patient for entering an email address, password, and password confirmation. The sign-up process can request a patient location zip code, or access to location services on a personal computing device, to aid in recommending treating professionals near a user's location.

The sign-up process will also instruct for a patient to construct a DHR that contains information relevant to the patient's identity and the patient's oral health. For example, the DHR can include, but is not limited to including, patient medical information items including x-rays, three-dimensional (3-D) models of a dental patient's mouth, and/or digital and/or analog pictures of the patient's smile. The DHR may also include other medical information, including current and past pharmaceutical prescriptions, health history, genomic information, etc. For patient identifying information, the DHR may include patient name, address, contact information (e.g., telephone number, fax number, electronic mail address), date of birth, gender, and/or dental insurance, among others. The DHR can also include personal treatment goals of the patient (e.g., gap closure, restoration, whitening) and the subscription payment status within the network (tier 1, tier 2, etc.).

The DHR may be filled in primarily by the patient or in concert between the patient and treating professionals. The patient may select a known treating professional with access to historical patient medical information or may select a new treating professional based on zip code or membership in the subscription network. Alternatively, the network platform may assign a treating professional based on skill, location, or other criteria. The selected or assigned treating professional may assist the patient with acquiring new patient medial information and images. With patient permission, the DHR can be updated to include prescription notes and/or treating professional comments in text, audio, and/or video clip format.

The DHR can be used to determine and/or consult on oral health. For example, the network platform may use the information in the DHR to rate overall oral health of the patient, or any individual aspect thereof. The network platform may also recommend care or resources for education based on the expected stage of life of the patient. For example, oral health recommendations for an eighteen-year-old patient undergoing orthodontic treatment can differ from those for a thirty-year-old patient having relapsed, making the age and history of the patient useful for providing education or selecting subsequent treating professionals. The rating and recommendation may occur automatically via dedicated algorithms running on a processor within the platform or may be done manually by an oral health expert.

Once the DHR is updated with requisite information, the patient may be connected via an app or other personal computing device interface to an oral health expert (the “concierge”). The concierge may be a doctor, dentist, orthodontist, dental hygienist, dental assistant, or any other professional capable of offering advice on any aspect of oral health. The concierge may communicate with the patient via text, chat window, encrypted instant message (SnapChat, WhatsApp, etc.), and/or telephone call. The concierge may either proactively reach out to the patient upon completion of the DHR or may await specific patient questions. The concierge may suggest treatment options based on the DHR and may answer questions regarding the length or complexity of treatment. The concierge can confirm patient interest and suitability for certain forms of treatment and can direct the patient to treating professionals within the network. The concierge may remain available to the patient throughout the life of the service, offering tips on preventative oral health (e.g., diet, floss, etc.) or treatment compliance. The concierge may also, with patient permission, offer reminders and other nudges to seek or maintain oral health treatment.

The concierge may connect with the patient's selected treating professional or may reach out to a network treating professional to acquire additional patient data to complete the DHR. The concierge may suggest patient data acquisition opportunities or office visit goals to either or both the treating professional and the patient. The concierge may request information on various dental treatments of interest to the patient from the treating professionals, including but not limited to cost and estimated time to completion.

The concierge or other node in the network may connect the patient with a dental supply retailer to supply dental and other oral health products to the patient. The retailer can be a co-branding partner that uses the brand name linked or suitably associated with the network such that users would not know that the network is actually operated by a third party. The retailer can offer dental products for brushing, flossing, and cleaning of dental implants and bridges. Other dental products could include anti-plaque rinse and plaque-fighting toothpaste. The retailer can also sell other health-care-related products such as prescription drugs; non-prescription drugs; personal care products; beauty and spa products; vitamins; herbs and nutrition; and medical supplies.

The patient may be offered several treatment options based on the DHR and/or treating professional feedback. The treatment options can include simulations of treatment using only photographs supplied by the patient, or based on more comprehensive dental imaging (e.g., xrays, digital scan, etc.) The patient's personal computing device may display a modified 2D representation of a patient's dentition. In some implementations, the modified 2D representation may include estimated results of an orthodontic treatment plan. The modified 2D representation may show a photo-realistic visualization of how application of an orthodontic treatment plan will appear on the patient. Additionally, or alternatively, the 2D representation may show the installation of an orthodontic appliance on the patient's current dentition. In other implementations, the patient personal computer may display a 3D representation of the user's face augmented with virtual appliances or treatment. Methods and systems for simulating treatment or appliance placement can be found, for example, in US Patent Publication US 20190350680 (Chekh et al.) and WO2019/215550 (Unklesbay et al.)

The treatment simulations and representations may further be tied to an estimated cost of treatment. The estimated cost may be based on case difficulty, appliance fabrication cost, patient location, patient treatment goals, number of available treating professionals in the network, estimated completion time, number of treatments requested, or any other factors that tend or may tend to influence ultimate cost to the patient. The patient may also be offered the opportunity to subscribe to additional tiers of the network to access additional features and treatment options.

The steps of the process for generating a treatment plan can be implemented as computer program modules for execution on one or more computer systems. Systems and methods for generating a treatment plan can be found, for example, in U.S. Pat. No. 7,435,083 (Chisti et al.), U.S. Pat. No. 7,134,874 (Chisti et al.), U.S. Patent Publication Nos. 2009/0286196 (Wen et al.); 2010/0260405 (Cinader) and U.S. Pat. No. 9,259,295 (Christoff et al.). The exemplary treatment plan described below is cast specifically to the treatment of a malocclusion with clear tray aligners (CTAs), but steps can be generalized for treatment with labial braces, lingual braces, and other dental treatments.

As an initial step, a mold, scan, or photograph of patient's teeth or mouth tissue is acquired. This generally involves direct contact scanning, x-ray imaging, tomographic imaging, sonographic imaging, taking cast, wax bite plates, and other techniques for obtaining information about the position and structure of the teeth, jaws, gums and other relevant tissue. A digital data set is derived from this data that represents an initial (e.g., pretreatment) arrangement of the patient's teeth and other tissues. A computer model of the arch may then be re-constructed based on the data.

Desired final positions of the teeth, or tooth positions that are desired and/or intended end result of orthodontic treatment, can be received, e.g., from a treating professional in the form of a descriptive prescription, can be calculated using basic orthodontic prescriptions, or can be extrapolated computationally from a clinical prescription. With a specification of the desired final positions of the teeth and a digital representation of the teeth themselves, the final position and surface geometry of each tooth can be specified to form a complete model of the teeth at the desired end of treatment or treatment stage. The result of this step is a set of digital data structures that represents a desired and/or orthodontically correct repositioning of the modeled teeth relative to presumed-stable tissue. The teeth and surrounding tissue can both be represented as digital data. Further details on software and processes that may be used to derive the target dental arrangement are disclosed, e.g., in U.S. Pat. No. 6,739,870 (Lai et al.), and U.S. Pat. Nos. 8,194,067; 7,291,011; 7,354,268; 7,869,983 and 7,726,968 (Raby et al.).

Having both a beginning position and a final target position for each tooth, the process can next define a treatment path or tooth path for the motion of each tooth. This can includes defining a plurality of planned successive tooth arrangements for moving teeth along a treatment path from an initial arrangement to a selected final arrangement. In one embodiment, the tooth paths are optimized in the aggregate so that the teeth are moved in the most efficient and clinically acceptable fashion to bring the teeth from their initial positions to their desired final positions. A movement pathway for each tooth between a beginning position and a desired final position may be calculated based on a number of parameters, including the total distance of tooth movement, the difficulty in moving the teeth (e.g., based on the surrounding structures, the types and locations of teeth being moved, etc.) and other patient-specific data that may be provided. Based on this sort of information, a user or a computer program may generate an appropriate number of intermediary steps (corresponding to a number of treatment steps). In some variations, the user may specify a number of steps, and the software can map different appliance configurations accordingly.

If the movement path requires that the teeth move more than a predetermined amount (e.g., 0.3 mm or less in X or Y translation), then the movement path may be divided up into multiple steps, where each step corresponds to a separate target arrangement. The predetermined amount is generally the amount that an appliance or appliance configuration can move a tooth in a particular direction in the time required for each treatment step. Each appliance configuration corresponds to a planned successive arrangement of the teeth and represents a step along the treatment path for the patient. For example, the steps can be defined and calculated so that each discrete position can follow by straight-line tooth movement or simple rotation from the tooth positions achieved by the preceding discrete step and so that the amount of repositioning required at each step involves an orthodontically optimal amount of force on the patient's dentition.

At various stages, the process can include interaction with a treating professional or concierge responsible for the treatment of the patient. Practitioner interaction can be implemented using the network platform and any connected participant. In some or all embodiments, the treatment planning may be embodied within a computer-readable storage medium, such as a computer-readable storage medium of clinician's computing device and/or manufacturer's computer, or both. The computer-readable storage medium stores computer-executable instructions that, when executed, configure a processor to perform the model preparation and treatment planning techniques described above.

The plurality of successive dental arrangements may be incorporated into a single appliance or apportioned between multiple appliances to be worn in series. Accordingly, a suitable treatment plan identifies a number of appliances in an acceptable series, as well as a target arrangement and a commencing arrangement for each appliance in the series. A plurality of planned, successive arrangements may be stored between the target and the commencing arrangements. As defined herein, the “target arrangement” may be a desired final dental arrangement or a planned successive dental arrangement the patient should reach after treatment with the appliance. In contrast, the “commencing arrangement” is the dental arrangement the appliance is configured to represent when the appliance is first placed in the patient's mouth. As such, it is closest in orientation to the initial or current arrangement of the patient's teeth, and in some embodiments represents the current arrangement.

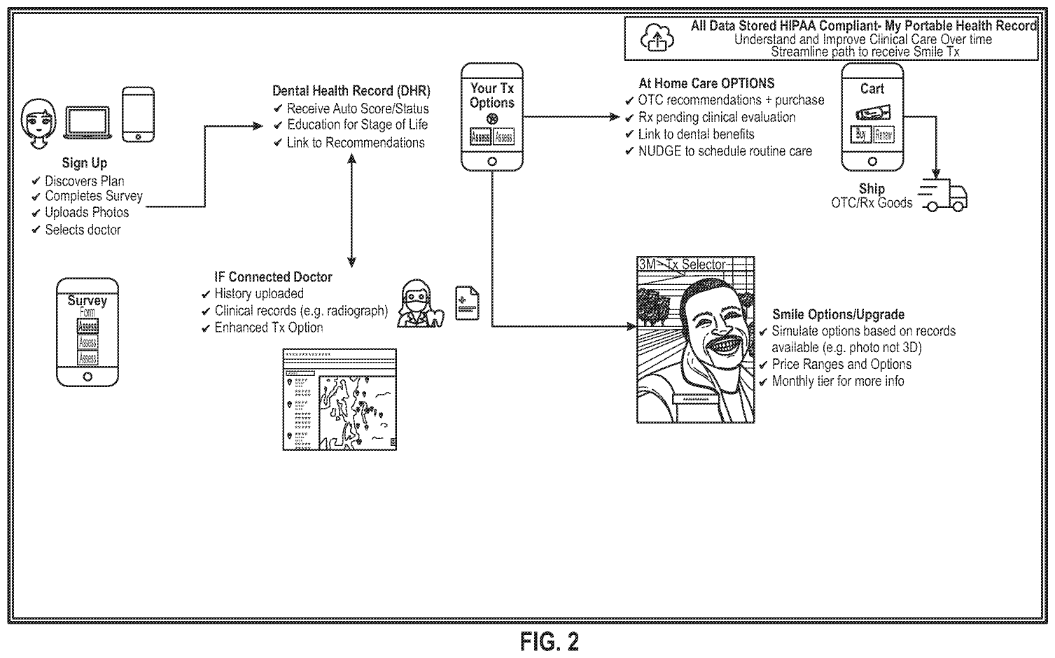

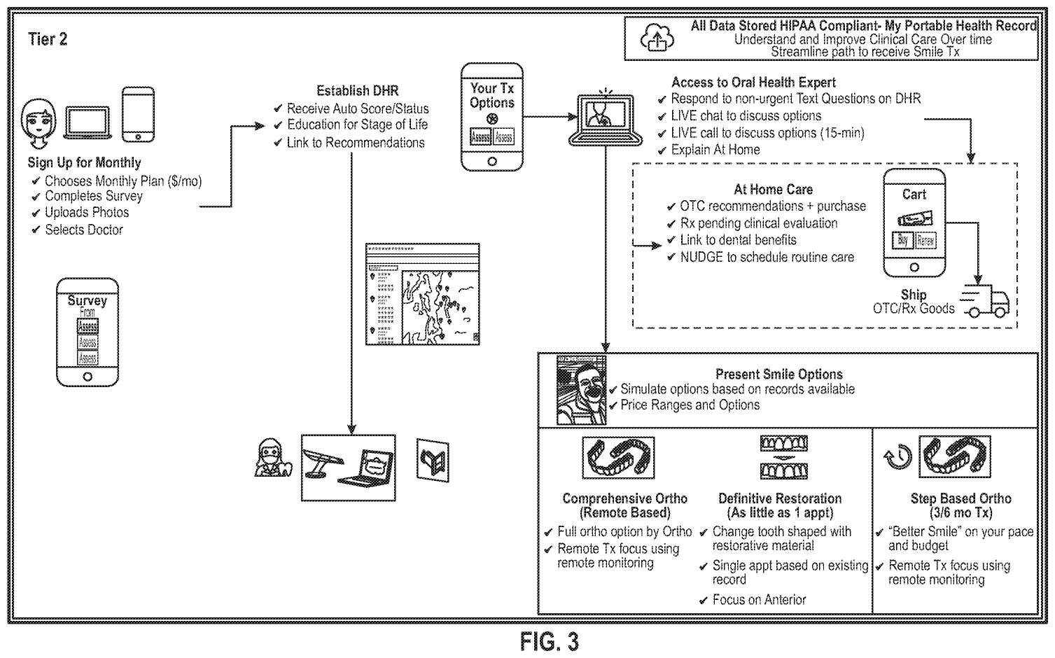

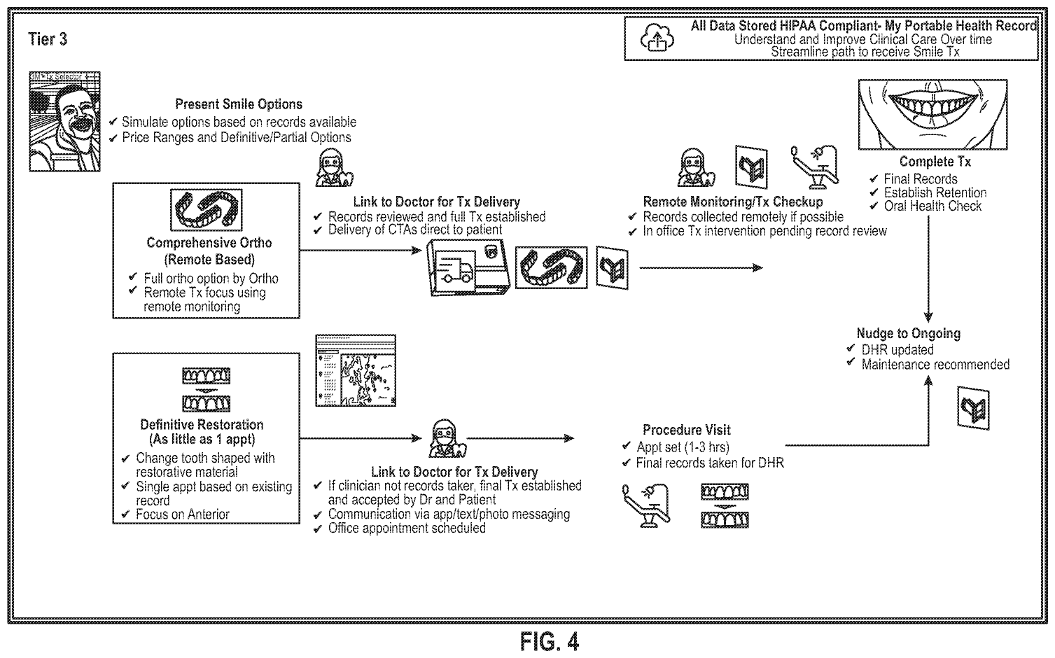

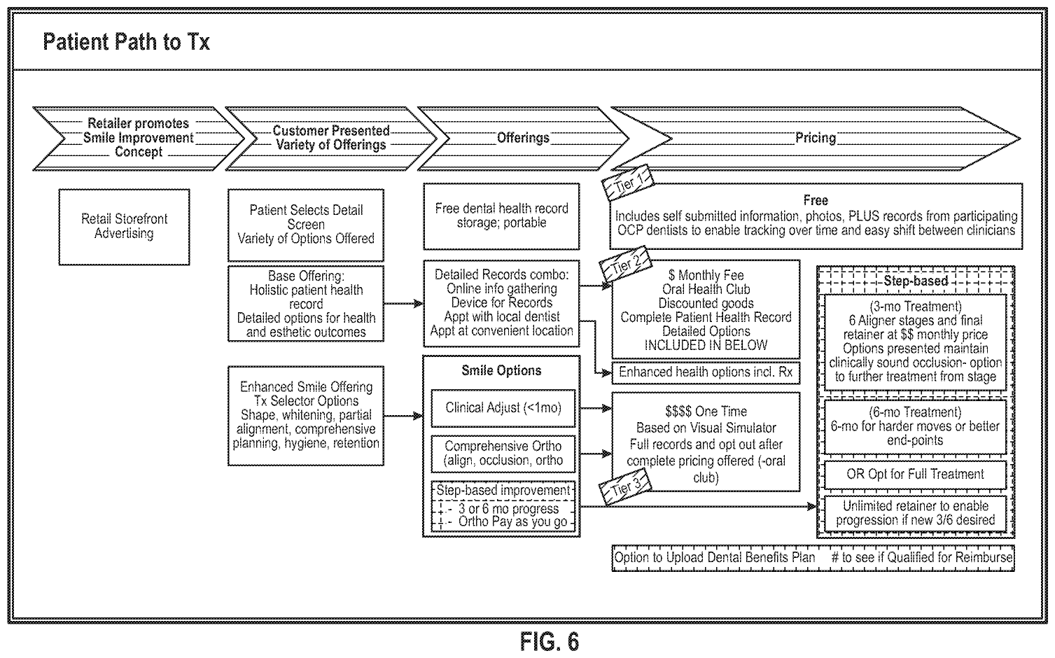

The network may include at least three tiers of subscription, with higher tiers offering more features and treatment options. FIG. 2 illustrates a free option, designated Tier 1. FIG. 3 illustrates a second tier, designated Tier 2, that is premised on patient payment of a recurring (e.g., monthly) fee. FIGS. 4 and 6 illustrate a premium tier, designated Tier 3, that is premised on the patient paying a higher monthly fee or agreeing to comprehensive treatment.

Tier 1 offers the patient the opportunity to compile a patient portable, patient controlled DHR, order dental supplies from one or more retail partners, review treatment simulations without committing to further consultation, and explore educational resources for oral health. Tier 1 may offer the treating professional a means to communicate more naturally with the patient, as well as share (with patient permission) the DHR with other specialists to consult on or directly provide patient treatment. The patient may choose to upgrade to a higher subscription tier at any time.

Tier 2 offers all the services and features of Tier 1 with the add benefit of access to a concierge. The benefits of Tier 2 may also include discounts on oral health products from one or more retail partners, volume-based discounts from network treating professionals, and other economic incentives to frequently engage in oral health. Tier 2 subscribers may be offered the choice of payment on any recurring basis, including monthly, quarterly, or yearly. Tier 2 subscribers may pay upfront or on a graduated basis. The fees may be distributed amongst the network participants in any fashion. In some embodiments, the fees are paid to a network administrator that may be a dental and orthodontic appliance manufacturer and distributed to other participants based on care or purchase metrics. In other embodiments, the fees are collected by the retail partner and distributed to participants based on product purchases, case starts, or number of appliances manufactured.

A financing entity connected to the network can offer patients one or more electronic financing options to pay for the higher tier subscription. In one implementation, the financing entity can be a credit card processing company or bank. The patient may enter the sensitive data such as credit card number, shipping address, among others, onto the DHR or other purchase form. The credit data is then submitted, collected and passed securely through the network platform. This data can be processed in real-time online or can be collected by mail or telephone and then entered by an operator. If the credit card information is deemed valid, the subscription price will be reserved from the issuing bank of the consumer's credit card and allocated to the account associated with the treatment. Alternatively, the financing entity can debit the subscription costs from the customer's checking account over the Internet. The information is processed as an Electronic Funds Transfer (EFT) to the customer's account using an Automated Clearinghouse (ACH) payment system.

Tier 2, like Tier 1, can offer patients several treatment plans for consideration and selection. These plans may be devised by treating professional based on the DHR, may be automatically generated by a server within the network platform, or may be provided by a dental product/appliance manufacturer, possibly in collaboration with other doctors, technicians, or service providers within the network. Appliances or prostheses may be created and applied to the patient's natural anatomy, and the patient's naturally anatomy can be augmented or modified as part of the treatment plan. 3D virtual models of such appliances and prostheses can be created by, for example, third party labs or service providers and combined with the datasets provided by the doctor. Modified anatomies resulting from treatment can be generated by the treating professional, the concierge, by a third party, or as a collaborative effort using remote software collaboration tools. The treatment plan can be revised based on the DHR or concierge, presented back to the patient for review, possible modification, and approval. In some scenarios, the treating professional may be the reviewer, and a third party lab or manufacturer is the source of the proposed treatment plan. The treatment plan may also include representations or predictions of the patient's future dentition in the absence of the selected or recommended treatment.

The combination of two or more of the DHR, concierge, and the subscription fees allows the network to provide essentially pre-qualified patients to treating professionals. The patient can have at least some introductory questions answered and treatment options considered. Subscribing patients will have demonstrated an ability to pay for at least some orthodontic and/or dental treatment. The network can also better match patients and treating professional based on a more wholistic view of the patient's life and health thanks to the shared DHR.

Tier 3 offers comprehensive treatment options based on the developed DHR. The comprehensive treatment may include orthodontic treatment, restorative treatment, or both. The comprehensive treatment may further include additional preventive or retentive treatments, or any other treatment regime designed to enhance oral health. If a tier 3 subscriber selects a comprehensive orthodontic treatment, the request can be promptly sent to a selected (either by the patient or concierge) treating professional to the review the patient DHR and assemble a treatment plan. The treating professional may independently create the treatment plan, may consult with other treating professionals to create the treatment plan, or may approve a treatment plan created by an appliance manufacturer.

Once the treatment plan is approved, patient may authorize payment via the financing entity. Alternatively, and depending on the recurring fee paid and the scope of the treatment plan, the treatment may be covered by the subscription. The authorized treatment plan can either result in the appliance manufacturer creating appliances to effectuate the treatment plan or the treating professional creating the appliances in a “chair side” process. Regardless of source, the appliances may be shipped directly to the patient. The appliances may be provided with a remote monitoring kit to assist the treating professional in tracking the progress of treatment. The remote monitoring kit may include a dental image acquisition support attachable to a patient's mobile phone to assist in the acquisition of dental images. Dental image support devices can be found, for example, in International Publication No. WO2020/089248 (Salah et al.) and as commercially available from GetGRIN, Inc.

After wearing an appliance for a prescribed period of time, the patient may reengage with the treating professional who may evaluate the result of the first iteration of treatment. In the event that the first iteration of treatment has resulted in satisfactory final placement of the patient's teeth, the treatment may be ended. However, if the first iteration of treatment did not complete the desired movement of the patient's teeth, one or more additional iterations of treatment may be performed. To begin the next iteration of treatment, the treating professional may take another scan of the patient's teeth or request another remote dental image from the patient's personal computing device to facilitate the design of the ordered set of removable dental appliances. In some examples, evaluation of the result of the first iteration of treatment may include taking another scan of the patient's teeth, in which case beginning the next iteration of treatment may simply involve forwarding the digital model of the patient's teeth to a manufacturing facility so that another appliance or series of appliances may be manufactured for the patient based on the new positions of the patient's teeth. In yet other examples, the newly acquired scan may be used to create one or more iterations of appliances in the treating professional's facility.

Because a patient's teeth may respond differently than originally expected, the treating professional may wish to evaluate the patient's progress during the course of treatment. The system can also do this automatically, starting from a newly measured in-course dentition. If the patient's teeth do not progress as planned, the treating professional can revise the treatment plan in the DHR as necessary to bring the patient's treatment back on course or to design an alternative treatment plan. The treating professional may provide comments, oral or written, for use in revising the treatment plan. The treating professional may wish to limit initial appliance production, delaying production on subsequent appliances until the patient's progress has been evaluated and alignment configuration can be more accurately estimated.

The network may allow remote consultation with the treating professional once the appliances are received. The treating professional may remotely review images of the patient's dentition or request the patient's presence in at a treating professional facility. In one or more embodiments that utilize progressive treatment of a patient's teeth, second, third, or more intermediate digital representations of the teeth can be performed using any suitable technique or combination of techniques. In presently preferred techniques, at least a portion of the intermediate digital representation is created using a dental imaging device provided to the patient and acquired remote from the treating professional's facility. The practitioner or manufacturer can then utilize these intermediate representations to provide one or more additional appliances that are adapted to provide one or more corrective forces to the teeth such that one or more teeth are repositioned to either a subsequent intermediate arrangement or a final target arrangement. Any suitable technique or combination of techniques can be utilized to provide these intermediate scans, models, and arch members, e.g., the techniques described in U.S. Patent Application Publication No. 2010/0260405 (Cinader) and International Publication WO2016/109660 (Raby et al.).

Tier 3 subscribing patient may also have access to custom treatments and tools for restorative dentistry. Exemplary tools and methods for dental restoration/restorative dentistry are described in commonly-assigned patent applications United States Patent Publ. No. 2018/0021113, U.S. patent application Ser. No. 16/061,362, entitled “One-Piece Dental Restoration Molds,” filed Dec. 15, 2016, U.S. patent application Ser. No. 16/061,350, entitled “Dental Restoration Molds,” filed Dec. 15, 2016, International Patent Publication Nos. WO 2018/022616 and WO 2020/033532, each entitled “Dental Restoration Molds”, and International App. No. IB2020/054778, filed May 20, 2020 and entitled “Automated Creation of Tooth Restoration Dental Appliances”. Such tools can include a facial mold body for a patient-specific, customized fit with the facial side of at least one tooth of the patient, wherein the facial mold body includes a restorative portion, at least one aperture aligned with a portion of a surface of a tooth to be restored and at least one door having an inner surface forming a portion of the mold cavity encompassing missing tooth structure of the tooth to be restored.

The patient's DHR may be updated during and after treatment with new images, treatment goals, oral health care goals, etc.

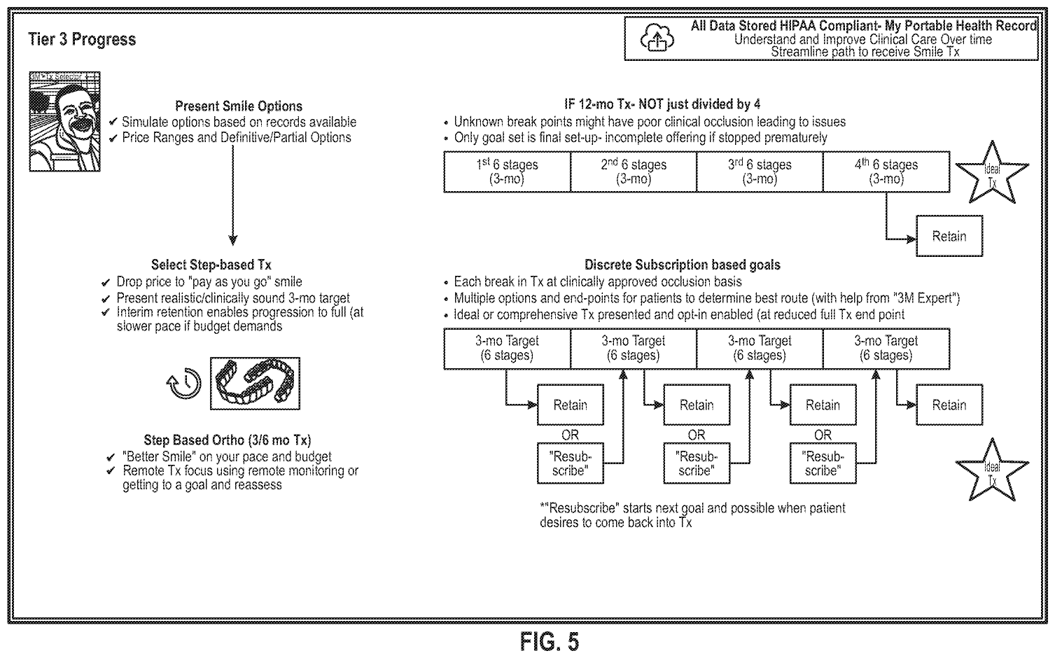

As illustrated in FIG. 5, tier 3 subscribers may choose to pay for treatment on a staged or monthly basis. Instead of focusing on comprehensive orthodontic treatment, the patient may choose to focus on a specific movement goal (e.g., gap closure in the anterior teeth) before deciding whether to continue with treatment. The patient may pay for a single stage of treatment lasting a limited (e.g., 3 month) duration that results in a clinically acceptable but not ideal resulting occlusion before choosing to proceed to another stage.

The successive stages of the treatment plan can be modified by the appliance manufacturer or treating professional to achieve clinically sound target arrangements at the end of a truncated stage of treatment. The treatment may be broken up in a set number of phases (i.e., stages) to allow for the termination, pause, or continued use of treatment at the end of each stage. The stages may be based on the severity of the occlusion and the patient treatment goals but will likely be less than a comprehensively realized treatment outcome. In one embodiment, the staged treatment proceeds on an opt-out basis, only terminating when expressly requested by the patient or treating professional. In another embodiment, the staged treatment proceeds on an opt-in basis, requiring affirmative consent from the patient or treating professional before progressing to a new stage. At the end of each stage in the opt-in version, the patient may choose to continue with one or more stages of treating by “re-subscribing” and paying for one or more additional stages. Alternatively, the patient may choose to pause or end treatment, at which point the patient can be provided with a retention appliance to preserve the dentition in the state reached at the end of treatment. Methods for retaining the position and orientation of teeth near the end of treatment (for either opt-out or opt-in treatment) may be found, for example, in U.S. Pat. No. 8,738,165 (Cinader et al.). At any point in time, the patient may choose to seek and pay for comprehensive treatment (e.g., the interactions of FIG. 4).

The approach above allows the patient to explore the benefits of orthodontic or other dental treatment without necessarily committing to a prohibitive up-front expenditure. It also allows for the introduction of new treatment options based on the changes to the patient's dentition as the staged treatment progresses through each new phase.

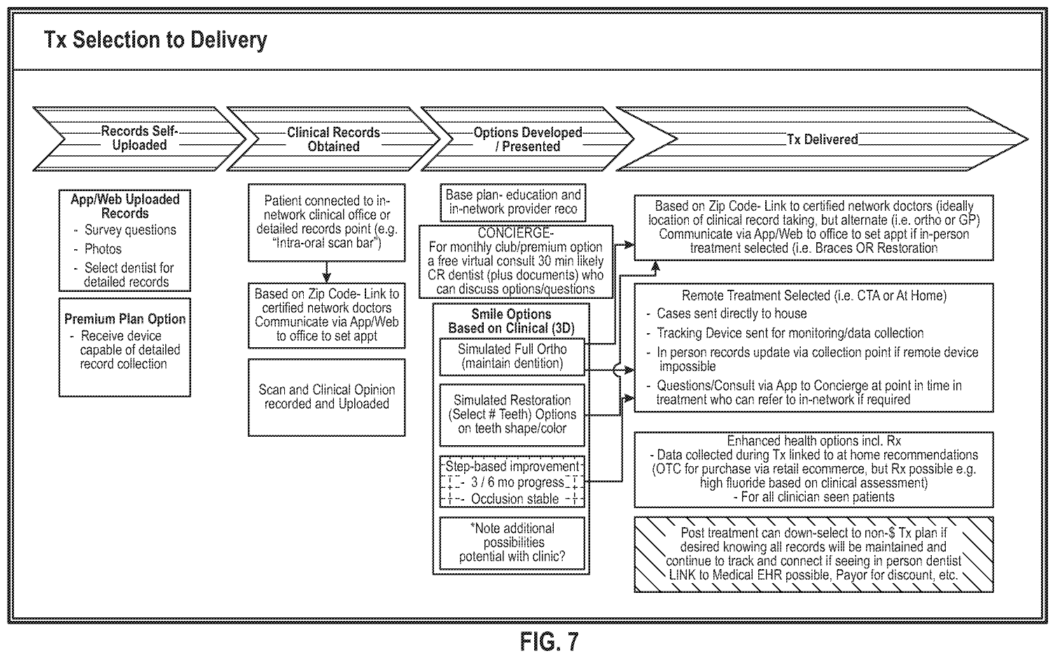

FIGS. 6 and 7 illustrate an exemplary usage of the network of FIG. 1, after the patient has signed up and logged in. The patient first selects a subscription tier, which dictates the scope and content of the offerings. Assuming the patient is a tier 2 or tier 3 subscriber, the patient is offered several treating professionals or is linked with his or her preferred professional. The patient is presented several treatment options, including pricing and payment options, based on information in the DHR and the subscription tier. If the patient has provided a personal photograph, the network platform can process the patient data in the DHR and render the patient's teeth in a plurality of alternative final states. The patient can also be offered other oral health care services, such as teeth whitening. After visualizing the effects of the potential treatments and reviewing pricing for the treatment, the patient may select one or more of the treatment options. Before or after the selection, the patient may be offered one or more financing options from a financial entity or appliance manufacturer.

The selection may be updated in the DHR, which is subsequently shared with one or more treating professionals. The treating professional(s) may review the DHR and decide to accept or decline the case. Upon acceptance of the case, the patient schedules a virtual or physical appointment with the selected treating professional. At the meeting, additional anatomical data and medical information can be captured from the patient by taking digital photographs of the face and teeth, taking x-rays of the front, back, side, and top/bottom of the patient, taking one or more impressions, etc. Next, certain or all of this information is entered into the DHR.

Once the patient requests treatment, a treating professional may generate or create a treating prescription or plan and send the prescription to an appliance manufacturing company. Appliances and delivery instruments may be sent directly to the patient's designated shipping address via the DHR. The patient may subsequently consult with a concierge or treating professional to begin treatment.

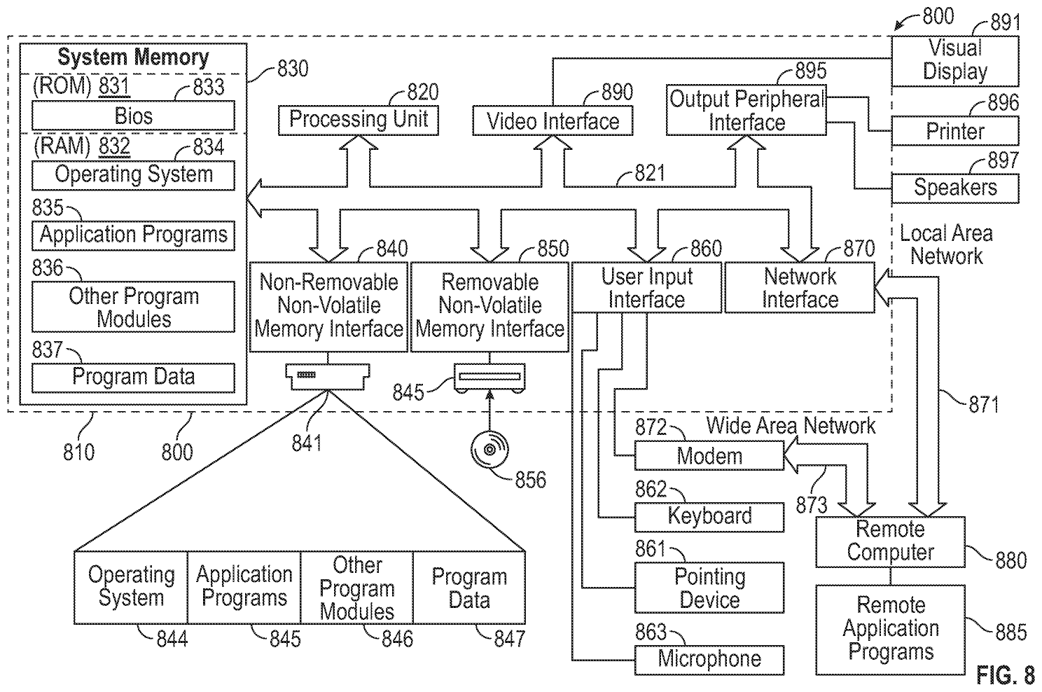

FIG. 8 is one example of a computing environment in which elements of systems and methods described herein, or parts of them (for example), can be deployed. With reference to FIG. 8, an example system for implementing some embodiments includes a general-purpose computing device in the form of a computer 810. Components of computer 810 may include, but are not limited to, a processing unit 820 (which can comprise a processor), a system memory 830, and a system bus 821 that couples various system components including the system memory to the processing unit 820. The system bus 821 may be any of several types of bus structures including a memory bus or memory controller, a peripheral bus, and a local bus using any of a variety of bus architectures. Memory and programs described with respect to systems and methods described herein can be deployed in corresponding portions of FIG. 8.

Computer 810 typically includes a variety of computer readable media. Computer readable media can be any available media that can be accessed by computer 810 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 810. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 830 includes computer storage media in the form of volatile and/or nonvolatile memory such as read only memory (ROM) 831 and random-access memory (RAM) 832. A basic input/output system 833 (BIOS) containing the basic routines that help to transfer information between elements within computer 810, such as during start-up, is typically stored in ROM 831. RAM 832 typically contains data and/or program modules that are immediately accessible to and/or presently being operated on by processing unit 820. By way of example, and not limitation, FIG. 8 illustrates operating system 834, application programs 835, other program modules 836, and program data 837. The computer 810 may also include other removable/non-removable and volatile/nonvolatile computer storage media. By way of example only, FIG. 8 illustrates a hard disk drive 841 that reads from or writes to non-removable, nonvolatile magnetic media, nonvolatile magnetic disk 852, an optical disk drive 855, and nonvolatile optical disk 856. The hard disk drive 841 is typically connected to the system bus 821 through a non-removable memory interface such as interface 840, and optical disk drive 855 are typically connected to the system bus 821 by a removable memory interface, such as interface 850.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc. The drives and their associated computer storage media discussed above and illustrated in FIG. 8, provide storage of computer readable instructions, data structures, program modules and other data for the computer 810. In FIG. 8, for example, hard disk drive 841 is illustrated as storing operating system 1844, application programs 845, other program modules 846, and program data 847. Note that these components can either be the same as or different from operating system 834, application programs 835, other program modules 836, and program data 837.

A user may enter commands and information into the computer 810 through input devices such as a keyboard 862, a microphone 863, and a pointing device 861, such as a mouse, trackball or touch pad. Other input devices (not shown) may include a joystick, game pad, satellite receiver, scanner, or the like. These and other input devices are often connected to the processing unit 820 through a user input interface 860 that is coupled to the system bus but may be connected by other interface and bus structures. A visual display 891 or other type of display device is also connected to the system bus 821 via an interface, such as a video interface 890. In addition to the monitor, computers may also include other peripheral output devices such as speakers 897 and printer 896, which may be connected through an output peripheral interface 895.

The computer 810 is operated in a networked environment using logical connections, such as a Local Area Network (LAN) or Wide Area Network (WAN) to one or more remote computers, such as a remote computer 880. When used in a LAN networking environment, the computer 810 is connected to the LAN 871 through a network interface or adapter 870. When used in a WAN networking environment, the computer 810 typically includes a modem 872 or other means for establishing communications over the WAN 873, such as the Internet. In a networked environment, program modules may be stored in a remote memory storage device. FIG. 8 illustrates, for example, that remote application programs 885 can reside on remote computer 880.

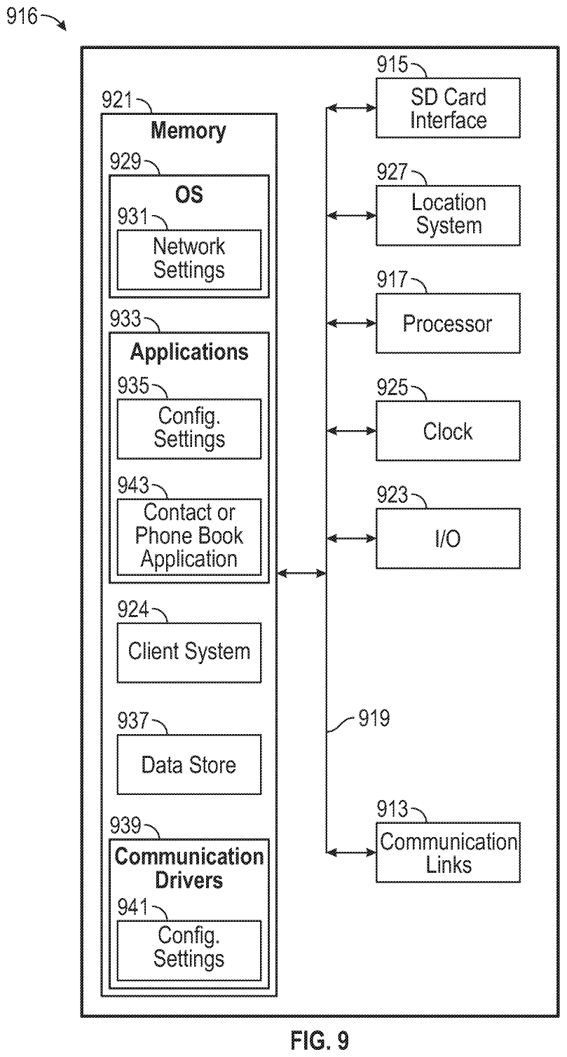

FIG. 9 illustrates an example mobile device that can be used in the embodiments shown in previous Figures. FIG. 9 is a simplified block diagram of one illustrative example of a handheld or mobile computing device that can be used as a user's or practitioner's handheld device, for example, in which the present system (or parts of it) can be deployed. For instance, a mobile device can be deployed in the operator compartment of computing device for use in generating, processing, or displaying the DHR, Tier Subscription, or Treatment Plan.

FIG. 9 provides a general block diagram of the components of a mobile cellular device 916 that can run some components shown and described herein. Mobile cellular device 916 interacts with them or runs some and interacts with some. In the device 916, a communications link 913 is provided that allows the handheld device to communicate with other computing devices and under some embodiments provides a channel for receiving information automatically, such as by scanning. Examples of communications link 913 include allowing communication though one or more communication protocols, such as wireless services used to provide cellular access to a network, as well as protocols that provide local wireless connections to networks.

In other examples, applications can be received on a removable Secure Digital (SD) card that is connected to an interface 915. Interface 915 and communication links 913 communicate with a processor 917 (which can also embody a processor) along a bus 919 that is also connected to memory 921 and input/output (I/O) components 923, as well as clock 925 and location system 927.

I/O components 923, in one embodiment, are provided to facilitate input and output operations and the device 916 can include input components such as buttons, touch sensors, optical sensors, microphones, touch screens, proximity sensors, accelerometers, orientation sensors and output components such as a display device, a speaker, and or a printer port. Other I/O components 923 can be used as well.

Clock 925 illustratively comprises a real time clock component that outputs a time and date. It can also provide timing functions for processor 917.

Illustratively, location system 927 includes a component that outputs a current geographical location of device 916. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 921 stores operating system 929, network settings 931, applications 933, application configuration settings 935, data store 937, communication drivers 939, and communication configuration settings 941. Memory 921 can include all types of tangible volatile and non-volatile computer-readable memory devices. It can also include computer storage media (described below). Memory 921 stores computer readable instructions that, when executed by processor 917, cause the processor to perform computer-implemented steps or functions according to the instructions. Processor 917 can be activated by other components to facilitate their functionality as well.

All of the patents and patent applications mentioned above are hereby expressly incorporated into the present description. The foregoing invention has been described in some detail by way of illustration and example for purposes of clarity and understanding. However, various alternatives, modifications, and equivalents may be used and the above description should not be taken as limiting in the scope of the invention which is defined by the following claims and their equivalents.

1. A subscription-based oral health network including one or more patients, one or more treating professionals, one or more appliance manufacturers, and one or more retail partners, wherein the patients are each provided a portable dental health record and a number of dental treatment options free of charge, and wherein payment of a recurring fee offers access to oral health consulting services and aesthetic oral care treatment.

2. The oral health network of claim 1, wherein the portable dental health record comprises at least two patient medical information items including x-rays, three-dimensional models of the patient's mouth, pictures of the patient's smile, current and past pharmaceutical prescriptions, health history, genomic information, personal treatment goals, contact information, and a subscription payment status.

3. The oral health network of claim 2, wherein the portable dental health record is a digital record stored on a server accessible by the patient and one or more treating professionals.

4. The oral health network of claim 1, wherein the network includes a platform including a processor adapted to communicate with a network; a data storage device coupled to the processor and adapted to store data for each patient; and software to communicate patient data in response to a patient, treating professional, or service provider request.

5. The oral health network of claim 4, wherein the network includes one or more oral health experts, wherein the expert provides tools to aid the patient in compiling a portal dental health record and includes access to a computing device in communication with a service provider through the platform.

6. A method of orthodontic treatment comprising a least two phases, wherein the patient is asked, via software application accessed via processor on a personal computing device, to opt-in to further treatment at the end of each phase.

7. The method of claim 6, wherein the patient must pay for further treatment at the end of each phase.

8. The method of claim 7, wherein the end of each phase represents a clinically acceptable occlusion or clinically acceptable treatment terminating juncture.
